(19) United States
(12) Patent Application Publication
(10) Pub. No.: US 2025/0195781 A1
(43) Pub. Date: Jun. 19, 2025

(54) DISPOSABLE PEN INJECTOR WITH INTEGRATED LOGGING USING NFC COMMUNICATION

(71) Applicant: Becton, Dickinson and Company, Franklin Lakes, NJ (US)

(72) Inventors: Bo Yang YU, Winchester, MA (US); J. Richard GYORY, Sudbury, MA (US); Kepei SUN, Andover, MA (US)

(73) Assignee: Becton, Dickinson and Company, Franklin Lakes, NJ (US)

(21) Appl. No.: 18/844,293

(22) PCT Filed: Mar. 10, 2022
(86) PCT No.: PCT/US2022/019679

§ 371 Date: Sep. 5, 2024

(51) Int. Cl. A61M 5/315; A61M 5/31

(52) U.S. Cl. CPC A61M 5/31568; A61M 2005/3126; A61M 2205/0233; A61M 2205/3592

(57) ABSTRACT

Disclosed are systems and methods for a computerized framework that electronically manages and controls a patient's therapy for proper administration of diabetic treatment. The framework includes a disposable pen injector that has integrated therein dose logging capabilities (e.g., a NFC circuit) for controlling the administration of the prescribed therapy. Electronic communications between the disposable pen injector and a therapy control application executing on a patient's device can effectuate accurate and efficient administration of prescribed therapy. The pen can communicate information to the patient's device, and the application executing thereon, that includes dosage information related to availability left in the pen, timing of doses, and the like. In a related manner, the application can compile dosage planning information that can dictate types of doses, amounts of doses, timing of doses, and the like, to ensure that the patient is complying with a prescribed diabetic prescription.

This application includes material that is subject to copyright protection. The copyright owner has no objection to the facsimile reproduction by anyone of the patent disclosure, as it appears in the Patent and Trademark Office files or records, but otherwise reserves all copyright rights whatsoever.

### FIELD OF THE DISCLOSURE

The present disclosure relates to a disposable pen injector, and more particularly, a disposable pen injector communicatively coupled with a device hosted application configured with functionality for dynamically controlling and managing injections of medication in a patient.

### BACKGROUND

Diabetes is a group of diseases marked by high levels of blood glucose resulting from defects in insulin production and/or insulin action. Currently, there are 37.3 million people in the United States, or 11.3% of the population, who have diabetes. Diabetes can lead to serious complications and premature death.

Treatment options for diabetes include specialized diets, oral medications and/or insulin therapy, which have the primary goal of controlling the patient's blood glucose (sugar) levels.

### SUMMARY

In accordance with one or more embodiments, a method is disclosed for a disposable pen injector to communicatively couple with a device hosted application that enables dynamic control, management and administration of therapeutic injections in a patient.

According to some embodiments, the method involves a device establishing an electronic connection with an injector pen, where the electronic connection enables the communication of information between the device and the injector pen, and the injector pen is configured to administer an injection of medication into a patient. The device then receives, via the electronic connection, first status information of the injector pen that indicates a current position of a plunger within a body of the injector pen and a time stamp. The first status information is analyzed by the device, which then determines a recommended dose of the medication that includes a timing in the future for administering a quantity of the medication. The device then communicates the information related to the recommended dose to the patient.

According to some embodiments, the method further involves the device receiving second status information of the injector pen, where the second status information indicates a position of the plunger within the body of the injector pen at a time associated with administration of the recommended dose.

In some embodiments, the method further involves the device analyzing the second status information based on the first status information, and then determining by the device, whether the recommended dose was properly administered via the injector pen.

In some embodiments, the device outputs a notification to the patient based on a determination of the administration of the recommended dose.

In some embodiments, the device monitors communication from the injector pen, where the reception of the second status information is based on the monitoring.

In some embodiments, the positions of the first and second status information are based on locations of detected conductive traces from conductive patterns on the plunger.

In some embodiments, the recommended dose further includes information related to a type of the medication.

In some embodiments, the method further involves the device communicating information related to the recommended dose to the injector pen via the electronic connection, and then causing, based on the communication, an adjustment of dose capabilities on the injector pen in accordance with the quantity of the recommended dose.

In some embodiments, the communication includes a display of the recommended dose on a display of the device.

In some embodiments, the information is communicated at time that corresponds to the timing of the recommended dose.

In some embodiments, the electronic communication is Near Field Communication (NFC), and the injector pen is configured with a NFC circuit.

In accordance with one or more embodiments, the present disclosure provides a non-transitory computer-readable storage medium for carrying out the above mentioned technical steps of improved materialized view matching. The non-transitory computer-readable storage medium has tangibly stored thereon, or tangibly encoded thereon, computer readable instructions that when executed by a device, cause at least one processor to perform a method for a disposable pen injector to communicatively couple with a device hosted application that enables dynamic control, management and administration of therapeutic injections in a patient.

In accordance with one or more embodiments, a system is provided that includes one or more computing devices and/or apparatus (e.g., a disposable pen injector) configured to provide functionality in accordance with such embodiments. In accordance with one or more embodiments, functionality is embodied in steps of a method performed by at least one computing device and/or apparatus. In accordance with one or more embodiments, program code (or program logic) executed by a processor(s) of a computing device to implement functionality in accordance with one or more such embodiments is embodied in, by and/or on a non-transitory computer-readable medium.

In accordance with one or more embodiments, an injector pen is disclosed. In some embodiments, the injector pen includes a plunger and a pen body.

In some embodiments, the plunger includes a printed conductive pattern on at least one side of the plunger.

In some embodiments, the pen body includes a metal spring, a voltage collection terminal (VCC), an analog-to-digital converter (ADC) and a NFC circuit. According to some embodiments, the metal spring is configured for interaction with the printed conductive pattern. In some embodiments, the VCC connects the metal spring and the ADC. In some embodiments, the ADC is configured to receive analog conductive traces via the VCC from interactions between the metal spring and printed conductive pattern and convert them to digital information. And, in some embodiments, the NFC circuit is configured to receive the digital data from the ADC, and communicate the digital data to an external device via a NFC connection.

Additional and/or other aspects and advantages of the present disclosure will be set forth in the description that follows, or will be apparent from the description, or may be learned by practice of the disclosure.

### BRIEF DESCRIPTION OF THE DRAWINGS

The features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 is a block diagram of an example configuration within which the systems and methods disclosed herein could be implemented according to some embodiments of the present disclosure;

FIG. 2 is a block diagram illustrating components of an exemplary system according to some embodiments of the present disclosure;

FIGS. 3A-3C illustrate exemplary embodiments of a disposable pen injector according to some embodiments of the present disclosure;

FIG. 4 illustrates an exemplary data flow according to some embodiments of the present disclosure; and

FIG. 5 is a block diagram illustrating a computing device showing an example of a device used in various embodiments of the present disclosure.

### DETAILED DESCRIPTION OF EXAMPLE EMBODIMENTS

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or” or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

Unless limited otherwise, the terms “connected,” “coupled,” “mounted” and variations thereof herein are used broadly and encompass direct and indirect connections, couplings, and mountings. In addition, the terms “connected” and “coupled” and variations thereof are not restricted to physical or mechanical connections or couplings. Further, terms such as “up,” “down,” “bottom,” “top,” “front,” “rear,” “upper,” “lower,” “upwardly,” “downwardly” and other orientational descriptors are intended to facilitate the description of the exemplary embodiments of the present disclosure, and are not intended to limit the structure of the exemplary embodiments of the present disclosure to any particular position or orientation. Terms of degree, such as “substantially” or “approximately,” are understood by those skilled in the art to refer to reasonable ranges around and including the given value and ranges outside the given value, for example, general tolerances associated with manufacturing, assembly, and use of the embodiments. The term “substantially,” when referring to a structure or characteristic, includes the characteristic that is mostly or entirely present in the characteristic or structure.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

For the purposes of this disclosure a non-transitory computer readable medium (or computer-readable storage medium/media) stores computer data, which data can include computer program code (or computer-executable instructions) that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router (WR) mesh, or 2nd, 3rd, 4th or 5th generation (2G, 3G, 4G or 5G) cellular technology, mobile edge computing (MEC), Bluetooth, 802.11b/g/n, or the like. Network access technologies may enable wide area coverage for devices, such as client devices with varying degrees of mobility, for example.

In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For purposes of this disclosure, a client (or consumer or user) device, referred to as user equipment (UE)), may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

A client device (UE) may vary in terms of capabilities or features. Claimed subject matter is intended to cover a wide range of potential variations, such as a web-enabled client device or previously mentioned devices may include a high-resolution screen (HD or 4K for example), one or more physical or virtual keyboards, mass storage, one or more accelerometers, one or more gyroscopes, global positioning system (GPS) or other location-identifying type capability, or a display with a high degree of functionality, such as a touch-sensitive color 2D or 3D display, for example.

The principles of the disclosed embodiments of the disclosed systems and methods will now be discussed in more detail.

By way of background, diabetes treatments, including multiple daily injections (MDI), insulin pens (with or without the applicable pen injectors), patch pumps and insulin pumps, are not configured with capabilities and/or functionality for automatically determining and recording information related to medication doses delivered to the patient. Existing mechanisms for providing diabetes treatments rely on historical logs of manually entered data most typically provided by the patient (and/or, in some cases, a health care provider (HCP)). That is, for example, daily records for (1) insulin dosing, (2) medication intake and available reservoir, and (3) Blood Glucose Measurement (BGM) can currently only be obtained from sources limited to settings on an insulin pen, an episodic reading from a BGM meter, and the estimate of carbohydrates in a meal all determined and transposed by the patient into a logbook or diary.

These methods of recording data are extremely tedious and prone to errors and omissions. For example, injecting a wrong dose and/or wrong type of prescribed insulin, or failing to take a proper dose (if at all) can lead to severe health complications (e.g., a diabetic coma) and even death.

Current solutions require third party software to reconfigure manually entered data to evaluate trends and support therapeutic modifications. However, this is still reliant on a patient's logbook, which, even under ideal situations, is still subject to human error (e.g., miss reading current insulin levels, administering a wrong dose or wrong type of medication, and the like). As a result, the risk of poor adherence to prescribed therapy, as well as imbalanced glycemic control, is elevated by such current solutions, and their reliance on the patient's diligent use in managing their prescribed therapy discredits their use as a viable option for management and control of diabetic therapy administration.

The systems and methods disclosed herein address these shortcomings, among others, and provide an improved computerized framework that electronically manages and controls a patient's therapy. According to some embodiments, the disclosed systems and methods are configured to automatically capture, store, analyze, update, transfer and enable optimal assessment of data necessary for the proper administration of diabetic treatment.

As evident from the discussion herein, the disclosed framework operates to eliminate the strict reliance on patient's logbooks via a novel disposable pen injector that has integrated therein dose logging capabilities for controlling the administration of the prescribed therapy for a patient. In some embodiments, the disclosed systems and methods operate via interaction between the disposable pen injector and an application (e.g., a therapy control application) executing on a patient's device (e.g., a smartphone, for example). As discussed below, the disposable pen injector is configured in a novel manner with a circuit that enables electronic connectivity and communication with the therapy control application. In some embodiments, as discussed below, the circuit integrated on the disposable pen injector can be, but is not limited to, a NFC circuit.

In some embodiments, as discussed in more detail below, a disposable pen injector is configured to provide the therapy control application with information related to, but not limited to, remaining volume from the disposable pen, information related to the last dose (or a set of previous doses) administered to the patient, type of dose present in the pen, and the like. As discussed in more detail below, upon reception of this data, which can be communicated from the pen via NFC functionality to the patient's device, the therapy control application can analyze the information and dynamically determine information related to a next dose, such as, but not limited to, a next dose amount and/or type, past, present and/or future dosing patterns, a timing for a prescription refill, available drug volume, and the like. In some embodiments, the therapy control application can also be configured to provide notifications or alarms to the user to alert them to a timing of a next dose, an improper dose, a missed dose, a timing for refilling a type of prescription, and the like, or some combination thereof

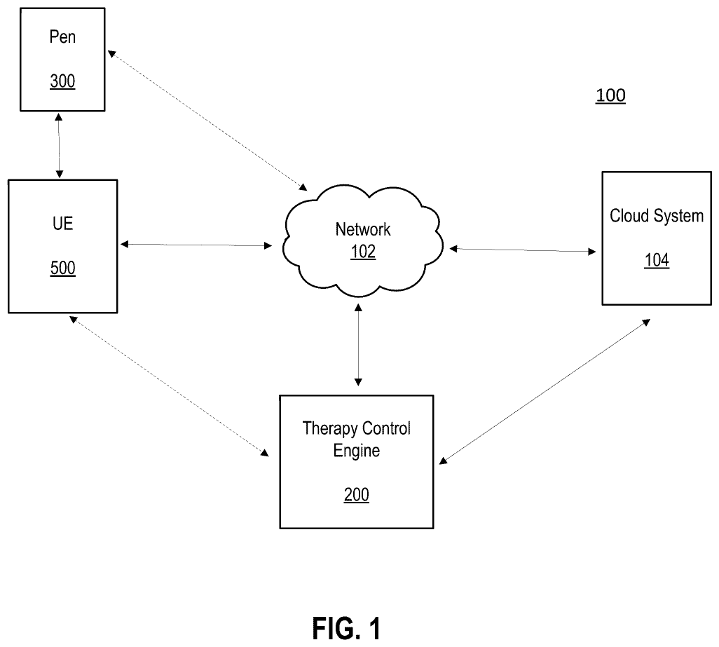

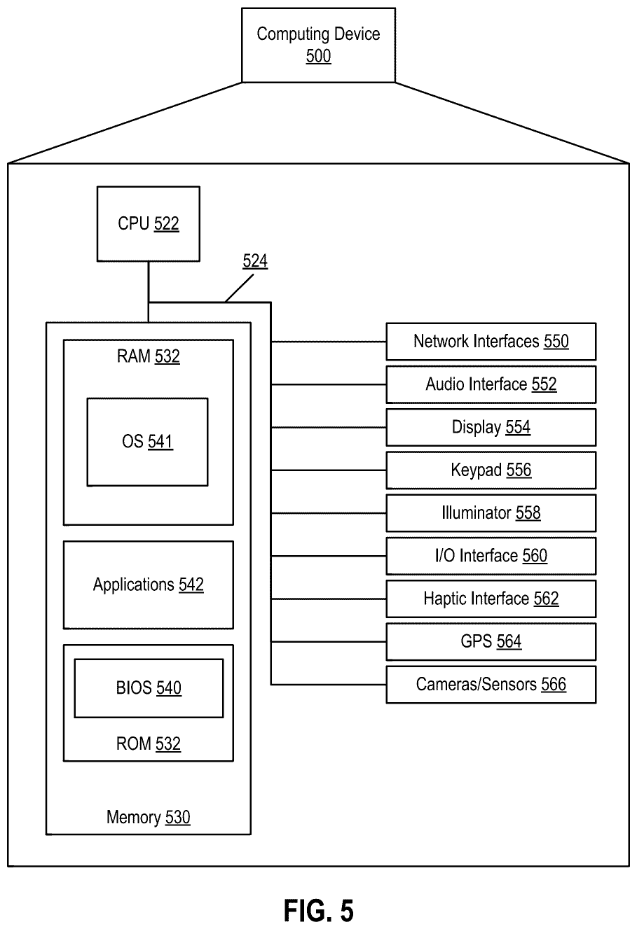

Turning to FIG. 1, system (or framework) 100 is depicted which includes UE 500 (e.g., a client device, as mentioned above), network 102, cloud system 104 and therapy control engine 200. UE 500 can be any type of device, such as, but not limited to, a mobile phone, tablet, laptop, personal computer, sensor, Internet of Things (IoT) device, autonomous machine, and any other device equipped with a cellular or wireless or wired transceiver. Further discussion of UE 500 is provided below in reference to FIG. 5.

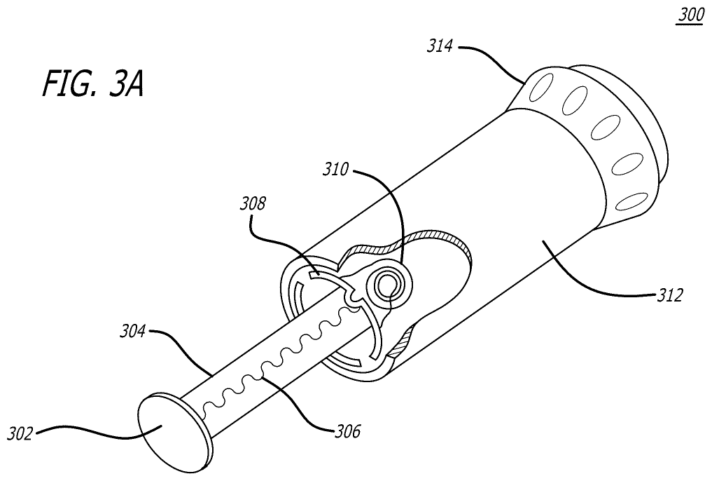

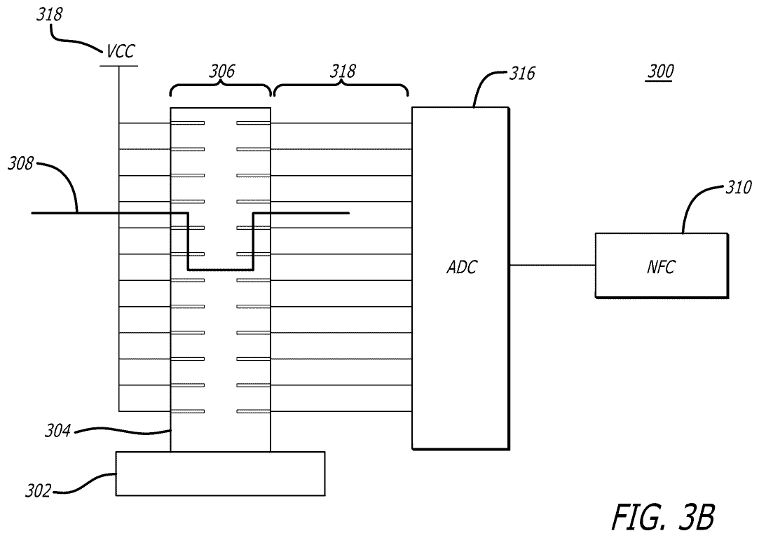

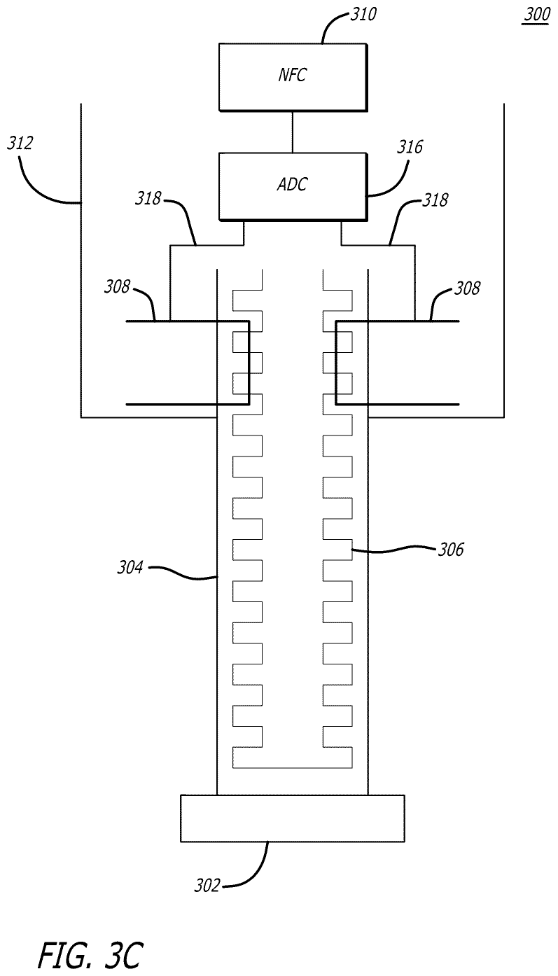

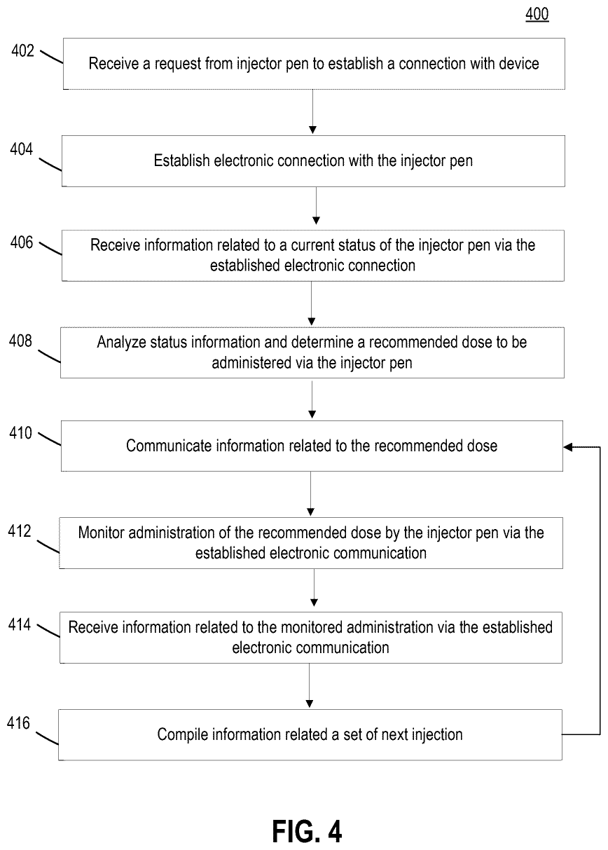

As illustrated in FIG. 1, UE 500 can be communicatively connected to a pen injector 300 (also referred to as a injector pen, medication pen, insulin pen or pen, used interchangeably). Pen 300 can communicate with UE 500 directly via a set of communication protocols that enable two electronic devices to interact over a set of distances. For example, as discussed below at least in relation to FIGS. 3A-4, pen 300 can be configured with a NFC circuit that enables NFC connectivity with UE 500. According to some embodiments, other forms of distance based communications (as discussed above), such as, but not limited to, RF, IR or Bluetooth Low Energy (BLE), in addition to any other type of known or to be known communication protocol can be used without departing from the scope of the instant disclosure.

In some embodiments, pen 300 can communicate with UE 500 via network 102 (as indicated by the dashed line connecting pen 300 and network 102 in FIG. 1). Network 102 can be any type of network, such as, but not limited to, a wireless network, cellular network, the Internet, and the like (as discussed above). As discussed herein, network 102 can facilitate connectivity of the components of system 100, as illustrated in FIG. 1. Thus, for example, in some embodiments, pen 300 can establish a wireless connection via network 102 with UE 500.

Pen 300 is a device used for injecting or administering medication under the skin of a patient (e.g., a human or person). As understood by those of skill in the art, injector pens, such as pen 300, are commonly used for medications that are injected repeatedly by a person over a relatively short period of time, for example, insulin and insulin analogs used in treatment of diabetes.

It should be understood that while the discussion herein will focus on embodiments directed to insulin pens, it should not be construed as limiting, as a pen injector (e.g., pen 300) can be used for any type of administration of medication related to, but not limited to, high cholesterol, migraine prevention and other monoclonal antibodies in relation to other types of known or to be known metabolic disorders and prescribed therapies.

Non-limiting embodiments of configurations and functionality of pen 300 are discussed below at least in relation to FIGS. 3A-4.

Cloud system 104 can be any type of cloud operating platform and/or network based system upon which applications, operations, and/or other forms of network resources can be located. For example, system 104 can be a service provider, network provider and/or medical provider from where services and/or applications can be accessed, sourced or executed from. In some embodiments, cloud system 104 can include a server(s) and/or a database of information which is accessible over network 102. In some embodiments, a database (not shown) of cloud system 104 can store a dataset of data and metadata associated with local and/or network information related to a user(s) of UE 500 and the UE 500, and the services and applications provided by cloud system 104 and/or therapy control engine 200.

Therapy control engine 200, as discussed below in more detail, includes components for communicating information between UE 500 and pen 300, and dynamically determining and dictating therapeutic control of prescribed medication to a patient. That is, for example, engine 200 can enable UE 500 to receive insulin dose information from pen 300, determine a prescribed compliance and schedule according to a prescription, and effectively manage the usage of the pen 300 by a patient, as discussed below.

According to some embodiments, therapy control engine 200 can be a special purpose machine or processor and could be hosted by a device on network 102, within cloud system 104 and/or on UE 500. In some embodiments, engine 200 can be hosted by a peripheral device connected to UE 500.

According to some embodiments, therapy control engine 200 can function as an application provided by cloud system 104. In some embodiments, engine 200 can function as an application installed on UE 500. In some embodiments, such application can be a web-based application accessed by UE 500 over network 102 from cloud system 104 (e.g., as indicated by the connection between network 102 and engine 200, and/or the dashed line between UE 500 and engine 200 in FIG. 1). In some embodiments, engine 200 can be configured and/or installed as an augmenting script, program or application (e.g., a plug-in or extension) to another application or program provided by cloud system 104 and/or executing on UE 500.

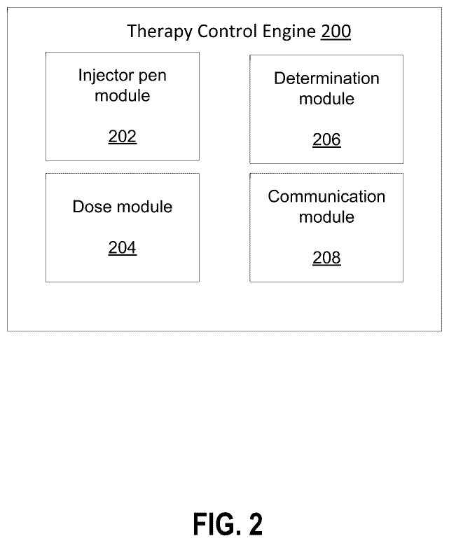

As illustrated in FIG. 2, according to some embodiments, therapy control engine 200 includes injector pen module 202, dose module 204, determination module 206 and communication module 208. It should be understood that the engine(s) and modules discussed herein are non-exhaustive, as additional or fewer engines and/or modules (or sub-modules) may be applicable to the embodiments of the systems and methods discussed.

More detail of the operations, configurations and functionalities of engine 200 and each of its modules, and their role within embodiments of the present disclosure will be discussed below in relation to FIGS. 3A-4.

FIGS. 3A-3C illustrate non-limiting embodiments of pen 300. For example, pen 300 can be a pen injector, such as, but not limited to, a BD Vystra™ Pen.

According to some embodiments, FIG. 3A illustrates a view of pen 300 and components included therein for performing the capabilities and functionality discussed herein; FIG. 3B illustrates a non-limiting example of configuration and operation of components of pen 300; and FIG. 3C illustrates another non-limiting example of configuration and operation of components of pen 300.

Turning to FIG. 3A, pen 300 includes plunger button 302, plunger 304, printed (or etched) conductive pattern 306, leaf springs 308 (e.g., metal contacts), NFC circuit (or chip or antenna) 310, pen body (or chamber) 312, and dose selection dial 314.

In some embodiments, the plunger button 302 can be configured as part of the plunger 304 that is designed to fit and move within and pen body 312. The plunger button 302 can be configured to enable a patient to depress the plunger 304 through and within the pen body 312 to inject a dose, for example, of insulin into a patient. The plunger 304 can act as a piston configured at a proximal end of the pen body 312, with the dose selection dial 314 located at the distal end. The plunger 304 can enable the capture and output of medication housed within the pen body 312 through the distal end via a hollow cannula or needle (not shown).

In some embodiments, the plunger 304 can be configured as an auto-injector, which may not require the patient (or user) to press the plunger button 302 to inject the dose. As discussed below, such automatic injection can be controlled via information received via NFC circuit 310 from a therapy control application executing on a connected device (e.g., UE 500, as discussed above in relation to FIG. 1).

In some embodiments, a conductive (or resistive) pattern 306 can be printed on the body of the plunger 304, as illustrated in FIG. 3A. In some embodiments, the conductive pattern 306 can include a conductive ink that is created via infusing graphite or other conductive materials into ink and then printing them onto the plunger 304. The printed conductive pattern 306, therefore, provides a conductive (or resistive) trace that indicates the location or depth of the plunger 304 respective to the pen body 312 (e.g., how depressed is the plunger 304 within the body 312, and/or an indication of the movement of the plunger 304 within the body 312).

In some embodiments, the conductive pattern 306 can alternatively be a product of copper or plated substrate etching, or any other known or to be known imprinting or etching technique where a conductive/resistive measurement or trace can be obtained from a plunger within a body of a disposable pen (e.g., RFID tags, a circuit scribe, and the like, for example).

In some embodiments, the conducive pattern 306 can be detected by metal contacts positioned with the pen body 312, referred to as leaf springs 308. In some embodiments, the leaf springs 308 can be metal, or another material, that is capable of interacting with the conductive pattern 306 and provide a voltage, current or other form of conductive/resistive trace capable of being read, as discussed more in detail below.

In some embodiments, plunger 304 can be configured with a predetermined set of leaf springs 308 in order to interact with (e.g., via friction with the pattern 306) and detect the conductive/resistive traces from the conductive pattern 306 on the plunger 304. In some embodiments, the number of leaf springs 308 can correspond to a number of patterns 306 on the plunger 304. For example, if plunger 304 has two conductive patterns 306 that are 180 degrees apart on the plunger 304, then there may be two (2) leaf springs 308 positioned within the pen body 312 (as illustrated in FIG. 3C) to interact with the patterns 306 positioning on the plunger 304.

In some embodiments, the conductive traces detected by the leaf springs 308 can be fed or transmitted to the NFC circuit 310. More details of this are discussed below in relation to FIGS. 3B and 3C.

In some embodiments, pen body 312 can be a chamber or can include a cartridge (or other type of reservoir) where medication can be housed. As discussed above, by passing the plunger 304 through the body 312, the medication can be injected into a patient. In some embodiments, the pen body 312 can be configured to house the medication. In some embodiments, for reusable versions of pen 300, pen body 312 can be refillable; and in some embodiments, the pen body 312 can be configured to house a replaceable cartridge, which can be loaded and unloaded into the pen body 312 for reuse of the pen 300 itself.

In some embodiments, dose selection dial 314 can be configured to enable accurate dose measuring, whereby it can be adjusted via radial adjustment of the dial in accordance with measurements indicated on the dial 314. In some embodiments, the dial 314 can include a clicking sound or other known or to be known method to confirm a dose adjustment.

Turning to FIG. 3B, a view of pen 300 is provided, which is a schematic internal view of pen 300, which includes plunger button 302, plunger 304, printed conductive pattern 306, a leaf spring 308, NFC circuit 310, analog-digital converter (ADC) 316 and a voltage collection terminal (VCC) 318.

According to some embodiments, as plunger 304 travels through pen body 312, whereby the conductive pattern 306 and leaf spring 308 interact with each other so as to register a conductive/resistive trace that is detected and communicated to ADC 316 by VCC 318. The communication of the detected conductive/resistive traces by VCC 318 to ADC 316 enables the detection of movement and/or location of the plunger 304, which, as discussed above and in more detail below, can indicate a remaining volume of medication, amount of a dose given, and the like, or some combination thereof.

According to some embodiments, the conductive/resistive trace that can be detected and registered by the VCC 318 in FIG. 3B, provides voltage information to the ADC 316. ADC 316, which is configured to act as a digital input/output (DIO) interface for NFC circuit 310, receives the analog voltage data, and converts it to digital data, which it then passes to NFC circuit 310. In some embodiments, ADC 316 can be configured as an integrated circuit within pen body 312 that is coupled to NFC circuit 310, and enables NFC circuit 310 to understand the analog readings being taken from movements by the plunger 304.

As mentioned above, and discussed below in more detail at least in relation to FIG. 4, digital data passed to NFC circuit 310 can then be communicated to a connected device (e.g., UE 500, as discussed above in relation to FIG. 1). Moreover, the connected device (e.g., UE 500) can pass calibrated information to pen 300 via NFC circuit 300, as discussed below. Further discussion of the conductive/resistive data captured, analyzed, compiled and utilized will be discussed in more detail below.

In FIG. 3C, another non-limiting view of pen 300 is provided, which is a schematic internal view of pen 300 that includes the components discussed above in relation to FIGS. 3A and 3B. FIG. 3C illustrates two leaf springs 308 situated at positions to interact with two conductive patterns 306 along the opposite sides of plunger 304. Each leaf spring 308 has connected thereto, or positioned proximate to a conductive portion of a spring 308, a VCC 318 for detection of conductive/resistive traces from the leaf springs 308 interactions with a respective conductive pattern 306. Thus, each VCC 318, as illustrated in FIG. 3C, enables traces (e.g., analog voltages) to be fed to ADC 316, which then provides digital readings to NFC circuit 310.

Thus, the disclosed systems and methods, as illustrated in FIGS. 3A-3B and discussed in more detail below in relation to FIG. 4, can detect an initial position of a plunger 304 (e.g., before a dose is given), a subsequent position of the plunger 304 (e.g., after a dose is given), whether a dose was given (e.g., did the plunger move from a last position), a speed of movement of a plunger 304 (e.g., was a dose given in a proper manner—for example, depression of the plunger 304 at a speed beyond a threshold speed may indicate that the proper dose of medication was not captured and/or passed via injection to a patient), and the like, or some combination thereof. As discussed below, this information can be passed to and from the injector pen 300 and a therapy control application executing on a patient's device (UE 500), thereby enabling accurate and proper administration of a prescribed medication.

FIG. 4 provides Process 400 which details non-limiting example embodiments of a disposable pen injector being communicatively coupled with a device hosted application for dynamically controlling and managing injections in a patient.

According to some embodiments, Steps 402-404 and 412 can be performed by injector pen module 202 of therapy control engine 200; Steps 406 and 414 can be performed by dose module 204; Step 410 can be performed by communication module 208; and Steps 408 and 416 can be performed by determination module 206.

Process 400 begins with Step 402 where engine 200 receives a request associated with an injector pen to establish an electronic connection with a device. As discussed above at least in relation to FIGS. 1-3C, for example, engine 200 can correspond to a therapy control application executing on UE 500 (e.g., a patient's smartphone, for example), and the injector pen 300 can utilize an internal NFC circuit 300 to communicate with the UE 500.

In Step 404, in response to the request from Step 402, an electronic connection can be established between the injector pen and device. Thus, for example, Step 404 can involve pen 300 being at or within a predetermined distance to UE 500, whereby an NFC connection can be established between the two devices. According to some embodiments, any type of known or to be known methodology for establishing an NFC connection between at least two devices can be performed without departing from the scope of the instant disclosure.

In Step 406, engine 200 receives information related to a current status of the injector pen. The status information received is sent by pen 300, via NFC circuit 310, to the therapy control application executing on UE 500.

According to some embodiments, the status information can include, but is not limited to, a current position of the plunger 304 within the pen body 312 and a time stamp. As discussed below, this information can provide an indication as to how much medication is remaining in the pen, as well as a type of drug and the current time associated with the pen/plunger reading.

According to some embodiments, as discussed above at least in relation to FIGS. 3A-3C, the position within the conductive patterns 306 that the leaf springs 308 are interacting with can be the basis for the status information. That is, for example, where the leaf springs 308 are in contact with the conductive patterns 306 provides an indication of the positioning of the plunger 304.

In some embodiments, the contact by the leaf springs can provide a resistance measurement via VCC 318 to the NFC circuit 310, which is then communicated to the UE 500. In some embodiments, a measurement for the plunger 304 can be compiled by ADC 316 based on the information received by VCC 318. That is, the ADC 316 can output binary values for positions along the plunger (e.g., a preset intervals) that either indicate that the leaf springs are in contact with the pattern 306 (e.g., 1) or they are not (e.g., 0). By way of a non-limiting example, if the pattern 306 on plunger 304 has 5 detectable locations where a leaf spring 308 can be in contact, and the contact is at a position where half of the medication has been used, then the ADC can return a digital value string indicating: 00100, which indicates that the springs 308 are interacting with a middle section of the pattern 306 on the plunger 304.

In Step 408, having received the status information for the pen, engine 200 can analyze the status information and determine a recommended dose to be administered via the injector pen. The recommended dose includes information indicating how much of the medication to take (e.g., a quantity) and a timing of when to take the dose (in the future).

According to some embodiments, the analysis performed by engine 200 can involve any type of known or to be known machine learning (ML) and/or artificial intelligence (AI) analysis, including algorithms, techniques, classifiers and/or mechanisms such as, but not limited to, Bayesian network analysis, Hidden Markov Models, artificial neural network analysis, logical model and/or tree analysis, and the like.

According to some embodiments, Step 408's determination may not be limited to determining a recommended dose, as the determination can also include other forms of dose information, such as, but not limited to, how much of a medication remains in the pen, a type of medication, a pattern of dose administration, and the like or some combination thereof, as discussed above.

Thus, in some embodiments, Steps 406-408 can involve engine 200 receiving the status information of the pen (e.g., a current position of a plunger within an injector pen), and then determining when, how much and/or which type of dose the patient is to next administer via the pen. In some embodiments, the determined dose information of Step 408 can also or alternatively be related to a predicted pattern/schedule for the patient—that is, rather than simply determining a next dose, Step 408 can involve engine 200 determining a full schedule for the patient that complies with prescription information (e.g., take X amount of Y medicine every 4 hours for 10 days, for example).

In some embodiments, the determined dose information can be stored in an associated data storage for later retrieval and/or communication to the pen and/or other device or display screen for notification to a user. For example, the dose information can be stored locally on UE 500 and/or in a database associated with cloud system 104, as discussed above in relation to FIG. 1.

In Step 410, having determined the recommended dose for the patient, information related to this recommendation is communicated for use by the patient. In some embodiments, the communication can involve a display on a screen of the UE 500 (and/or in association with an interface of the therapy control application executing thereon) that indicates the recommended dose information (e.g., amount and/or timing). In some embodiments, the communication can involve a display on a peripheral device connected to UE 500 (e.g., a smartwatch connected to the patient's smartphone).

In some embodiments, the communication can be sent at a time of determination, at a time aligned with the recommended dose, at a time according to settings set by the patient, and the like, or some combination thereof.

According to some embodiments, in addition to the display of information, other forms of notifications can be additionally and/or alternatively communicated to the patient, which can include, but are not limited to, message (e.g., SMS or emails), sounds, vibrations (e.g., haptic effects), and the like, or some combination thereof.

In some embodiments, Step 410 can involve communicating the dose information directly to the injector pen via the NFC connection. According to some embodiments, this communication can enable the injector pen to automatically configure settings for administration of a next dose according to the recommended dose as determined in Step 408. For example, for injector pens with digital capabilities, the communication can cause dial 314 to be automatically adjusted according to a dose amount indicated by the recommended dose.

In Step 412, engine 200 monitors administration of the recommended (and communicated) dose via an established NFC connection with the injector pen. According to some embodiments, engine 200 can analyze incoming signals communicated by NFC circuit 310 of pen 300 and determine whether another dose has been administered. In some embodiments, this can involve determining if the plunger 304 has changed positions (which can be performed in a similar manner as discussed above in relation to Step 406).

In some embodiments, NFC circuit 310 may only be configured to send signals to UE 500 when the plunger 304 has moved (at least a threshold amount of distance to account for jostling and/or other types of involuntary movements). In such situations, the lack of received signals from the pen 300 can indicate to engine 200 that no such dose has been administered, and such monitoring of Step 412 can be continued.

In some embodiments, the monitoring of Step 412 can be in accordance with a timing of the recommended next dose. For example, if the next dose is recommended to be administered via pen 300 in 6 hours, then at a predetermined time proximate to the 6 hours (e.g., 10 minutes before, for example), engine 200 can begin periodically monitoring for NFC signals from pen 300.

In some embodiments, the monitoring in Step 412 (and Step 414, as discussed below) may require re-establishment of the NFC connection, such that the processing of Step 402-404 can be repeated to enable the monitoring and information reception discussed herein.

In Step 414, engine 200 receives information related to the administration of the recommended dose (e.g., an update of status information associated with injector pen 300). This information is received in a similar manner as discussed above in relation to Step 406. Accordingly, the information received in Step 414 can include, but is not limited to, a current position of the plunger 304 and a time stamp.

In Step 416, engine 200 compiles information related to a set of next injections (e.g., a next dose and/or a schedule of doses). This compilation can be performed in a similar manner as discussed above in relation to Steps 406-408.

In some embodiments, Step 416 can also involve determining whether an administered dose was properly administered. For example, the distance the plunger changed (from Step 406-414) can be analyzed and a determination can be made regarding whether the correct amount of medication was administered. In some embodiments, the time stamps can be analyzed as well and used to determine if the dose was administered at a proper time.

In some embodiments, the updated dose information compiled in Step 416 from the received information from Step 414 can be used to update the stored information for the patient (that was stored as a sub-process of Step 408, as discussed above). In some embodiments, such updating of the dose information for the patient can also include updating dosing pattern information, as discussed above.

According to some embodiments, engine 200 can be configured to periodically update stored dose information for a patient. That is, in some embodiments, rather than update dose information based on received dose information from pen 300, engine 200 can automatically update the dose information based on predicted/expected dosing. This can account for situations where NFC connections are unable to be enabled (e.g., the patient is not near his/her smartphone). Thus, engine 200 is capable of tracking a patient's compliance with a prescription as a safety measure to prevent improper dosing, improper injections, and the like.

Process 400 further involves recursively proceeding from Step 416 to Step 410 so as to track the administration of medicine according to the recommended dosing determined by the steps of Process 400.

FIG. 5 is a block diagram illustrating a computing device 500 (e.g., UE 500, as discussed above) showing an example of a client device or server device used in the various embodiments of the disclosure.

The computing device 500 may include more or fewer components than those shown in FIG. 5, depending on the deployment or usage of the device 500. For example, a server computing device, such as a rack-mounted server, may not include audio interfaces 552, displays 554, keypads 556, illuminators 558, haptic interfaces 562, GPS receivers 564, or cameras/sensors 566. Some devices may include additional components not shown, such as graphics processing unit (GPU) devices, cryptographic co-processors, artificial intelligence (AI) accelerators, or other peripheral devices.

As shown in FIG. 5, the device 500 includes a central processing unit (CPU) 522 in communication with a mass memory 530 via a bus 524. The computing device 500 also includes one or more network interfaces 550, an audio interface 552, a display 554, a keypad 556, an illuminator 558, an input/output interface 560, a haptic interface 562, an optional GPS receiver 564 (and/or an interchangeable or additional GNSS receiver) and a camera(s) or other optical, thermal, or electromagnetic sensors 566. Device 500 can include one camera/sensor 566 or a plurality of cameras/sensors 566. The positioning of the camera(s)/sensor(s) 566 on the device 500 can change per device 500 model, per device 500 capabilities, and the like, or some combination thereof.

In some embodiments, the CPU 522 may comprise a general-purpose CPU. The CPU 522 may comprise a single-core or multiple-core CPU. The CPU 522 may comprise a system-on-a-chip (SoC) or a similar embedded system. In some embodiments, a GPU may be used in place of, or in combination with, a CPU 522. Mass memory 530 may comprise a dynamic random-access memory (DRAM) device, a static random-access memory device (SRAM), or a Flash (e.g., NAND Flash) memory device. In some embodiments, mass memory 530 may comprise a combination of such memory types. In one embodiment, the bus 524 may comprise a Peripheral Component Interconnect Express (PCIe) bus. In some embodiments, the bus 524 may comprise multiple busses instead of a single bus.

Mass memory 530 illustrates another example of computer storage media for the storage of information such as computer-readable instructions, data structures, program modules, or other data. Mass memory 530 stores a basic input/output system (“BIOS”) 540 for controlling the low-level operation of the computing device 500. The mass memory also stores an operating system 541 for controlling the operation of the computing device 500.

Applications 542 may include computer-executable instructions which, when executed by the computing device 500, perform any of the methods (or portions of the methods) described previously in the description of the preceding Figures. In some embodiments, the software or programs implementing the method embodiments can be read from a hard disk drive (not illustrated) and temporarily stored in RAM 532 by CPU 522. CPU 522 may then read the software or data from RAM 532, process them, and store them to RAM 532 again.

The computing device 500 may optionally communicate with a base station (not shown) or directly with another computing device. Network interface 550 is sometimes known as a transceiver, transceiving device, or network interface card (NIC).

The audio interface 552 produces and receives audio signals such as the sound of a human voice. For example, the audio interface 552 may be coupled to a speaker and microphone (not shown) to enable telecommunication with others or generate an audio acknowledgment for some action. Display 554 may be a liquid crystal display (LCD), gas plasma, light-emitting diode (LED), or any other type of display used with a computing device. Display 554 may also include a touch-sensitive screen arranged to receive input from an object such as a stylus or a digit from a human hand.

Keypad 556 may comprise any input device arranged to receive input from a user. Illuminator 558 may provide a status indication or provide light.

The computing device 500 also comprises an input/output interface 560 for communicating with external devices, using communication technologies, such as USB, infrared, Bluetooth™, or the like. The haptic interface 562 provides tactile feedback to a user of the client device.

The optional GPS transceiver 564 can determine the physical coordinates of the computing device 500 on the surface of the Earth, which typically outputs a location as latitude and longitude values. GPS transceiver 564 can also employ other geo-positioning mechanisms, including, but not limited to, triangulation, assisted GPS (AGPS), E-OTD, CI, SAI, ETA, BSS, or the like, to further determine the physical location of the computing device 500 on the surface of the Earth. In one embodiment, however, the computing device 500 may communicate through other components, provide other information that may be employed to determine a physical location of the device, including, for example, a MAC address, IP address, or the like.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers, or be loaded and executed by one or more servers. One or more modules may be grouped into an engine or an application.

For the purposes of this disclosure the term “user”, “subscriber” “consumer” or “customer” should be understood to refer to a user of an application or applications as described herein and/or a consumer of data supplied by a data provider. By way of example, and not limitation, the term “user” or “subscriber” can refer to a person who receives data provided by the data or service provider over the Internet in a browser session, or can refer to an automated software application which receives the data and stores or processes the data.

Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

1. A method comprising the steps of:
establishing, by a device, an electronic connection with an injector pen, the electronic connection enabling the communication of information between the device and the injector pen, the injector pen configured to administer an injection of medication into a patient;
receiving, by the device, via the electronic connection, first status information of the injector pen, the first status information indicating a current position of a plunger within a body of the injector pen and a time stamp;
analyzing, by the device, the first status information, and determining, based on the analysis, a recommended dose of the medication, the recommended dose comprising a timing in the future for administering a quantity of the medication; and
communicating, by the device, information related to the recommended dose to the patient.

2. The method of claim 1, further comprising:
receiving, by the device, second status information of the injector pen, the second status information indicating a position of the plunger within the body of the injector pen at a time associated with administration of the recommended dose.

3. The method of claim 2, further comprising:
analyzing, by the device, the second status information based on the first status information; and
determining, by the device, whether the recommended dose was properly administered via the injector pen.

4. The method of claim 3, further comprising:
outputting, by the device, a notification to the patient based on a determination of the administration of the recommended dose.

5. The method of claim 2, further comprising:
monitoring, by the device, communication from the injector pen, wherein the reception of the second status information is based on the monitoring.

6. The method of claim 1, wherein the positions of the first and second status information are based on locations of detected conductive traces from conductive patterns on the plunger.

7. The method of claim 1, wherein the recommended dose further comprises information related to a type of the medication.

8. The method of claim 1, further comprising:
communicating, by the device, information related to the recommended dose to the injector pen via the electronic connection; and
causing, based on the communication, adjustment of dose capabilities on the injector pen in accordance with the quantity of the recommended dose.

9. The method of claim 1, wherein the communication comprises a display of the recommended dose on a display of the device.

10. The method of claim 1, wherein the information is communicated at time that corresponds to the timing of the recommended dose.

11. The method of claim 1, wherein the electronic communication is Near Field Communication (NFC), and wherein the injector pen is configured with a NFC circuit.

12. An injector pen comprising:
a plunger, the plunger comprising a printed conductive pattern on at least one side of the plunger; and
a pen body, the pen body comprising:
a metal spring configured for interaction with the printed conductive pattern;
a voltage collection terminal (VCC) connecting the metal spring and an analog-digital converter (ADC);
the ADC configured to receive analog conductive traces via the VCC from interactions between the metal spring and printed conductive pattern and convert them to digital information; and
a Near Field Communication (NFC) circuit, the NFC circuit configured to receive the digital data from the ADC, and communicate the digital data to an external device via a NFC connection.

13. A non-transitory computer-readable storage medium tangibly encoded with computer-executable instructions, that when executed by a device, perform a method comprising:
establishing, by the device, an electronic connection with an injector pen, the electronic connection enabling the communication of information between the device and the injector pen, the injector pen configured to administer an injection of medication into a patient;
receiving, by the device, via the electronic connection, first status information of the injector pen, the first status information indicating a current position of a plunger within a body of the injector pen and a time stamp;
analyzing, by the device, the first status information, and determining, based on the analysis, a recommended dose of the medication, the recommended dose comprising a timing in the future for administering a quantity of the medication; and
communicating, by the device, information related to the recommended dose to the patient.

14. The non-transitory computer-readable storage medium of claim 13, further comprising:
receiving, by the device, second status information of the injector pen, the second status information indicating a position of the plunger within the body of the injector pen at a time associated with administration of the recommended dose;
analyzing, by the device, the second status information based on the first status information;
determining, by the device, whether the recommended dose was properly administered via the injector pen; and
outputting, by the device, a notification to the patient based on a determination of the administration of the recommended dose.

15. The non-transitory computer-readable storage medium of claim 13, wherein the positions of the first and second status information are based on locations of detected conductive traces from conductive patterns on the plunger.

16. The non-transitory computer-readable storage medium of claim 13, further comprising:
communicating, by the device, information related to the recommended dose to the injector pen via the electronic connection; and
causing, based on the communication, adjustment of dose capabilities on the injector pen in accordance with the quantity of the recommended dose.

17. A device comprising:
a processor configured to:
establish an electronic connection with an injector pen, the electronic connection enabling the communication of information between the device and the injector pen, the injector pen configured to administer an injection of medication into a patient;
receive, via the electronic connection, first status information of the injector pen, the first status information indicating a current position of a plunger within a body of the injector pen and a time stamp;
analyze the first status information, and determine, based on the analysis, a recommended dose of the medication, the recommended dose comprising a timing in the future for administering a quantity of the medication; and
communicate information related to the recommended dose to the patient.

18. The device of claim 17, further comprising:
receive second status information of the injector pen, the second status information indicating a position of the plunger within the body of the injector pen at a time associated with administration of the recommended dose;
analyze the second status information based on the first status information;
determine whether the recommended dose was properly administered via the injector pen; and
output a notification to the patient based on a determination of the administration of the recommended dose.

19. The device of claim 17, wherein the positions of the first and second status information are based on locations of detected conductive traces from conductive patterns on the plunger.

20. The device of claim 17, further comprising:
communicate information related to the recommended dose to the injector pen via the electronic connection; and
cause, based on the communication, adjustment of dose capabilities on the injector pen in accordance with the quantity of the recommended dose.
